(19) United States
(12) Patent Application Publication
(10) Pub. No.: US 2026/0091209 A1
(43) Pub. Date: Apr. 2, 2026

(54) DRUG DELIVERY DEVICE

(71) Applicant: THE UNIVERSITY OF BIRMINGHAM, Birmingham (GB)

(72) Inventors: Christopher MCCONVILLE, Birmingham (GB); Manna AMIN, Birmingham (GB); Ali Al KOLAIB, Birmingham (GB)

(21) Appl. No.: 19/113,686

(22) PCT Filed: Sep. 19, 2023
(86) PCT No.: PCT/GB2023/052425

§ 371 Date: Mar. 20, 2025

(30) Foreign Application Priority Data
Sep. 20, 2022 (GB) 2213744.2

(51) Int. Cl. A61M 31/00; A61L 31/04; A61M 39/02

(52) U.S. Cl. CPC A61M 31/002; A61L 31/045; A61M 39/0208; A61M 2039/0235

(57) ABSTRACT

An implantable device (101) for drug delivery to a patient, the device comprising a housing (103) defining an enclosed space (106), a catheter port (102) that provides fluid access to the enclosed space, a permeable release zone (105) that permits drug release from the enclosed space, and an attachment portion (104) for attaching the housing at a location, such that, in use, the implantable device can be placed within the body of the patient and attached, using the attachment portion, at a location such that the release zone is adjacent to a treatment site, and whereby a drug composition in fluid form can be provided to the enclosed space via a catheter (108) and the catheter port, and the drug can be released to the treatment site via the permeable release zone.

### FIELD OF THE INVENTION

The present invention relates to the field of drug delivery to patients following surgery. More specifically, the present invention relates to an implantable device for drug composition delivery to a patient, a kit comprising such a device and a method of using such a device.

### BACKGROUND OF THE INVENTION

Five-year survival after surgery for pancreatic cancer is uncommon. In the UK, the five-year survival rate for all pancreatic cancers is 7%.

Patients with resectable pancreatic cancer undergo surgery to remove tumours. In the UK, the five-year survival rate for patients who undergo surgery to remove tumours is 20%. Surgery for pancreatic cancer is flawed because microscopic cancer is present at the surgical margin in most patients, correlating with both local recurrence and reduced survival. Local recurrence after surgery affects around half of all patients with locally invasive tumour growth being the cause of death in 30% of patients.

Combination chemotherapy has been shown to be increasingly effective following surgery to remove resectable pancreatic cancer. One particularly effective treatment is the FOLFIRINOX regime. This regime is a combination chemotherapy treatment for advanced pancreatic cancer which includes leucovorin, fluorouracil, ironitecan and oxaliplatin. A study by Conroy et al (2018) comparing FOLFIRINOX and gemcitabine demonstrated a median disease-free survival 21.6 and 12.8 months respectively. The disease-free survival rate at 3 years was 39.7% in the FOLFIRINOX group and 21.4% in the gemcitabine group. The median overall survival was 54.4 months in the FOLFIRINOX group and 35.0 months in the gemcitabine group. The overall survival rate at 3 years was 63.4% and 48.6% in the FOLFIRINOX and gemcitabine groups respectively. This difference in survival rates demonstrates the efficacy of this therapy and the importance of making it available to as many patients as possible.

However, up to half of all patients never receive any adjuvant chemotherapy, with even less receiving the more effective FOLFIRINOX regimen. Traditionally, chemotherapeutic drugs are administered systemically at high doses to ensure that the treatment penetrates deep into tissues. As chemotherapeutic drugs are highly toxic to the patient as well as the cancer, patients often need time to recover from surgery before undergoing chemotherapy. This is especially true for the FOLFIRINOX regime, which is highly toxic even by the standard set by chemotherapy. In the study by Conroy et al. comparing FOLFIRINOX with gemcitabine, adverse events of grade 3 or 4 occurred in 75.9% of the patients in the FOLFIRINOX group and in 52.9% of those in the gemcitabine group. Many patients cannot recover sufficiently to withstand systemic treatment with FOLFIRINOX within the treatment window of 6 to 8 weeks.

Local delivery of chemotherapeutic drugs to the surgical margin would reduce local recurrence of the cancer whilst allowing lower doses to be used. Lower dosage rates would reduce the negative impact of the chemotherapy on the patient, allowing treatment of weaker patients, which would make harsher treatments such as FOLFIRINOX available to more patients, and would reduce the time between surgery and chemotherapy, which would increase the efficacy of the treatment.

There is therefore a need for a device and method to deliver chemotherapeutic drugs directly to the surgical margin to reduce local recurrence without the associated toxicity of systemic delivery.

However, delivery of the drug to the surgical margin cannot be immediate. Following surgery patients can be weak and administration of toxic drugs could negatively impact their recovery. Furthermore, some drugs, in particular chemotherapeutic drugs can directly inhibit the healing of the surgical margin. The point in time following surgery at which a patient would be sufficiently recovered to receive chemotherapeutic treatment cannot be predicted ahead of surgery. Any method of post-surgery delivery of chemotherapeutic must therefore allow a medical professional to have control over the time at which the chemotherapeutic drugs are administered.

Several approaches exist for localised delivery of chemotherapy at the surgical margin following the removal of a pancreatic cancer tumour.

It has been disclosed in Xia et al., Advanced Health Materials 7:18 (2018) that gemcitabine-loaded electrospun fibres can be used for implantation at a resection site. The reference demonstrates that the fibres are more effective with reduced liver toxicity when compared to systemic administration of gemcitabine. However, the fibres begin release of gemcitabine from the moment that they are implanted. The health of the patient following surgery cannot be accurately determined prior to implanting the fibres, and such methods risk the patient's recovery as the moment that release begins is set before a medical professional can judge whether the patient is suitably recovered to receive the drugs.

It has also been disclosed in Byrne et al., Proc Natl Acad Sci USA (2016) that an implantable iontophoretic device is capable of driving chemotherapies deep into solid tumours. The drug continuously flows into and out of the device. This approach is essentially a chemo-wash, resulting in minimal exposure of the cancerous resection margin to the chemotherapeutic drug solution and requiring the patient to remain in hospital with a catheter and drain in place, increasing infection risk.

It is desirable for their own welfare and for the effective functioning of a healthcare system that any patient receiving chemotherapy is in hospital for as short a time as possible.

### SUMMARY OF THE INVENTION

The present inventor recognised the need for a device for delivery of a drug composition to a specific location within the body of a patient, such as at a surgical margin, wherein the time at which the drug composition is administered to the patient can be determined by a medical professional post-surgery.

The present invention provides, in a first aspect, an implantable device for drug delivery to a patient, the device comprising:
a housing defining an enclosed space:
a catheter port that provides fluid access to the enclosed space:
a permeable release zone that permits drug release from the enclosed space; and
an attachment portion for attaching the housing at a location:
   such that, in use, the implantable device can be placed within the body of the patient and attached, using the attachment portion, at a location such that the release zone is adjacent to a treatment site, and whereby a drug composition in fluid form can be provided to the enclosed space via a catheter and the catheter port, and the drug can be released to the treatment site via the permeable release zone.

The drug for delivery to the patient may be provided in the form of any drug composition that can flow. Thus, the drug composition may be considered to be a fluid. It is necessary that the drug composition can flow via a catheter and the catheter port into the enclosed space. The drug may be provided in the form of a liquid drug composition (e.g. a solution, suspension or emulsion), or in any other form that can flow, such as a free-flowing powder, or a semi-solid composition, e.g. a gel or paste or cream.

It is, in particular, envisaged that the drug could be provided in the form of a solution or a suspension. However, the invention is not limited to using such forms of drug composition.

The device can be equipped with a catheter (e.g. a Hickman Line). When the first end of the catheter is connected to the device via the catheter port, the catheter is in fluid communication with the enclosed space. When equipped with a catheter the device can be filled with fluid from a distance. Thus, the device can be located within the patient's body and a drug composition in fluid form can be provided from a location outside the patient's body.

Therefore, in a second aspect, the invention provides a kit comprising (i) the implantable device of the first aspect and (ii) a catheter.

The device can be implanted into the patient during or after a surgical procedure, in particular a surgical procedure to remove one or more tumours, such as a procedure to remove a resectable pancreatic cancer tumour. The treatment site can therefore be a resection site. It will be appreciated that it is convenient to implant the device during surgery, once the procedure has been completed but before the surgical opening has been closed.

The device can be placed within the body of the patient and attached, using the attachment portion, at a location such that the release zone is adjacent to a treatment site, e.g. in physical contact with a treatment site. The distal end of a catheter can be attached to the catheter port. The surgical opening can then be closed using standard surgical techniques, leaving the proximal end of the catheter protruding from the patient.

The catheter port and catheter allow fluid communication between the enclosed space of the housing and the outside of the patient's body.

Once a medical practitioner determines that the patient is ready to receive a drug (e.g. chemotherapy), the enclosed space within the housing can be filled with a drug composition in fluid form, such as liquid form, (e.g. a solution or suspension) via the catheter and catheter port.

Therefore, in a third aspect, the invention provides a method of facilitating drug administration to a patient, the method comprising the steps of:
a) providing an implantable device for drug delivery according to the first aspect:
b) placing the device within the body of the patient; and
c) attaching the device, using the attachment portion, at a location such that the release zone is adjacent to a treatment site.

In one embodiment, steps a) to c) are carried out during or after surgery on the patient, optionally wherein the surgery is surgery to remove one or more tumours. In one embodiment, the treatment site is a resection site.

The method may further comprise: i) providing a catheter; and ii) attaching the catheter to the catheter port: wherein steps i) and ii) can be carried out before or after any of steps a) to c). In one embodiment, step i) is carried out before step b) and step ii) is carried out before or after step b).

The method may further comprise:
d) providing a drug composition in fluid form to the enclosed space, via the catheter and the catheter port; and
e) allowing the drug to be released to the treatment site via the permeable release zone.

In one embodiment there is a controlled delay period between step c) and step d). For example, the delay period may be a day or more, or a week or more, or two weeks or more, or three weeks or more, or four weeks or more.

A significant benefit of the invention is that there is control over the timing of the drug delivery.

In this regard, there is a delay between attaching the device at a location within the body of the patient and providing the drug composition into the enclosed space of the housing via the catheter and catheter port. This delay can be controlled by the medical practitioner, and thus there can be a built-in delay period before the drug, e.g. chemotherapy, reaches the treatment site, e.g. resection site. In some embodiments this delay can be short, lasting only the time required to complete the surgery. However, in some embodiments the delay can be longer and chosen to give the patient a chance to become strong enough to receive the drug, e.g. to allow enough time to recover from the surgery. Recovery from the surgery may include healing of wounds, overcoming infections, improvement in overall strength and wellbeing, or any other parameter of recovery as determined by a medical professional. Importantly, the decision of when to transfer the drug composition into the enclosed space can be made by a medical professional based on facts available post-surgery. The medical professional is also able to modify the formulation of the drug composition, e.g. in terms of the drugs that are present, the concentration of drug used or any additional agents that are present, based on these facts.

The device therefore allows a medical professional to control when the delivery of the drug to the patient begins. If the patient is ready to receive the drug, the drug composition in fluid form can be transferred into the enclosed space via the catheter and catheter port. If the patient is too weak to receive the drug, or the medical professional deems that the surgical wound has not healed sufficiently, the transfer of the drug composition can be postponed.

There may optionally be a controlled delay period between step d) and step e). For example, the delay period may be a day or more, or a week or more, or two weeks or more, or three weeks or more, or four weeks or more. In other embodiments, however, step e) immediately follows step d), i.e. there is no delay, or any delay between step d) and step e) is less than 24 hours, such as less than 12 hours or less than 4 hours, e.g. less than 1 hour.

A further significant benefit of the invention is that is that there is control over the location of the drug delivery.

In this regard, the device allows localised drug delivery to a treatment site, e.g. a resection site, especially a surgical margin. The delivery of the drug is therefore specifically targeted to the required location. Lower dosage levels of the drug can therefore be used to obtain the same therapeutic effect without the side effects that are associated with systemic drug delivery.

The surgical opening can be closed around the proximal end of the catheter, such that the proximal end protrudes from the patient's body. The protrusion of this proximal end is unobtrusive and does not affect the patient's quality of life. Procedures where a catheter is left protruding from the body for days, weeks, or even months are well known. For example, biliary drainage catheters are usually in position for 8 to 12 weeks.

Once the device is installed the patient can leave the hospital and recover at home until it is time to administer the drug composition.

Once the drug composition has been transferred into the enclosed space, the catheter can be removed.

Once the drug composition has been transferred into the enclosed space, the patient is also able to leave the hospital whilst the drug is allowed to be released at the treatment site. This has benefits for the patient, who is able to go about their normal life before and after the drug composition is transferred. This also has benefits to a health care service, because the hospital bed is vacated quicker-meaning the health care capacity is not consumed by the procedure and the administration of the drug composition is cheaper.

In the present invention, the drug composition is transferred into the enclosed space of the device, via a catheter and the catheter port, and then the drug is allowed to be released to the treatment site via the permeable release zone.

In some embodiments, the permeable release zone releases the drug immediately to the treatment site. By immediately, it is meant that the permeable release zone does not substantially slow down the release of the drug to the treatment site. Therefore, it is released to the treatment site at substantially the same rate as if there was no physical barrier present.

In other embodiments, the permeable release zone releases the drug to the treatment site over a period of time. It is therefore released in a sustained manner. By a sustained manner, it is meant that the permeable release zone acts as a barrier and slows the release of the drug from the enclosed space when compared with an immediate release manner where there is no physical barrier. Thus, the release may take place over a period of time of an hour or more, e.g. 12 hours or more, or a day or more, e.g. two days or more, or a week or more, e.g. two weeks or more. Release in a sustained manner ensures that the release of the drug at the treatment site (e.g. at the surgical margin) is constant whilst at the same time reducing the overall peak concentration in the body.

A high peak concentration is associated with enhanced side effects. For example, in the case of the FOLFIRINOX regimen, high peak concentration can cause hair loss, severe diarrhoea, nausea, vomiting, constipation, loss of appetite, weight loss, infection, anaemia (which may require a blood transfusion), bruising, bleeding, headache, tiredness, weakness, dizziness, cough, shortness of breath, fever and pain, amongst other side effects.

In some embodiments, the permeable release zone is a perforated section of the housing, or a single opening in the housing, or a membrane extending across one or more openings in the housing.

In preferred embodiments the permeable release zone comprises, or is, a polymer membrane.

In some embodiments the polymer membrane is a sustained release membrane, configured to allow the transfer of drug in a sustained manner. The thickness of the membrane, number of perforations (e.g. pores) in the membrane, and size of perforations (e.g. pores) in the membrane can be controlled to ensure that the drug that has been transferred into the enclosed space is released over a sustained period, e.g. of hours, days, weeks, months or years.

Bursting of any implantable device containing liquid is undesirable for several reasons. A burst device no longer functions to release drugs to the treatment site at a controlled rate. A burst device immediately releases any drug compositions that are present in the enclosed space: this could have adverse health impacts for the patient, depending on the drug composition. Also, bursting of a device within a patient could cause damage/trauma at the burst site.

In some embodiments, therefore, the drug composition is in the form of a powder or a semi-solid, e.g. a gel or paste or cream. These forms of drug composition are less likely to lead to pressure on external walls of the device and therefore this reduces the risk of bursting.

In some embodiments, the drug composition is in liquid form and the implantable device comprises a sponge contained within the enclosed space. When the drug composition in liquid form is transferred to the enclosed space, the sponge absorbs the drug composition in liquid form and holds it within the enclosed space. Both the drug and other components of the drug composition such as a liquid carrier or diluent will be absorbed by the sponge in the enclosed space. Effectively, the sponge containing the drug composition can be considered as a solid content for the device. This arrangement is therefore also less likely to lead to pressure on external walls of the device, and therefore this option also reduces the risk of bursting.

A sponge is therefore contained within the enclosed space in certain preferred embodiments of the implantable device.

The skilled person will also appreciate that they can control what volume of drug composition is provided to the enclosed space. By adding a lower volume of drug composition, even if the drug composition is in liquid form then the device is still less likely to burst. Therefore in some embodiments the enclosed space contains no more than 75% by volume of drug composition, such as no more than 60% by volume, or no more than 50% by volume, or no more than 33% by volume.

The above-described product, kits and method allow safe and effective treatment of a patient with drug compositions, especially following surgery, such as surgery to remove one or more tumours. They have key benefits of being able to control the time at which the drug is provided to the patient and being able to control the exact location within the patient to which the drug is provided.

### DETAILED DESCRIPTION OF THE INVENTION

The implantable device is arranged such that a drug composition can be transferred to the enclosed space, via the catheter port, where it is held until the drug passes from the device into the body via the permeable release zone. The implantable device is held in position by the attachment portion. Therefore, the permeable release zone can be positioned adjacent to a treatment site in the patient's body, e.g., a resection site, such as a surgical margin.

This means there is control of the timing of the drug being released, because this cannot occur before the drug composition is transferred to the enclosed space, and in addition there may be further control built in due to the permeable release zone being configured to cause a delay in the release of the drug from the enclosed space into the patient's body.

There is also control in the location at which the drug is released, because the implantable device can be implanted at a chosen location in the patient's body, with the permeable release zone being positioned adjacent to the treatment site in the patient's body, thereby ensuring that the drug is provided directly to that treatment site.

The housing of the device is sized and shaped to be easily positioned within the body of a patient. In some embodiments, the housing has a substantially flat face which is provided with the catheter port. In this embodiment the top of the implantable device is defined as the face provided with the catheter port. The face provided with the catheter port may be referred to as the first face. The housing of the device may have a second face provided with the permeable release zone. This second face may be directly opposed to the first face and form the bottom of the device, or it may be positioned perpendicular to the first face, forming one or more side of the device. The second face may be directly attached to the first face, or it may be spaced apart from the first face by one or more wall, wherein the second and first face, together with the optional one or more wall, define the enclosed space.

In some embodiments the housing has a rectangular cross section. In other embodiments the housing may have a circular, semi-circular, oval, square, triangular cross section, or any other suitable cross section. In some embodiments any corners of the housing will be rounded.

The housing may have a rectangular cuboid shape, wherein optionally the corners of the housing are rounded. One or more of the faces of the rectangular cuboid housing may have a curved profile, such as a convex profile or a concave profile. The rectangular cuboid housing may have dimensions of less than 12 cm by less than 4 cm by less than 4 cm. For example, the rectangular cuboid housing may have dimensions of less than 10 cm by less than 3 cm by less than 3 cm or of from 12 to 4 cm by from 4 to 1 cm by 4 to 0.5 cm. A first face of the rectangular cuboid housing may be provided with the catheter port and a second face may be provided with the permeable release zone. The second face may be directly attached to the first face. The second face may be perpendicular to the first face. The first face provided with the catheter port may have dimensions of less than 4 cm by less than 4 cm, such as less than 3 cm by less than 3 cm or from 1 to 4 cm by from 0.5 to 4 cm, and the second face provided with the permeable release zone may have dimensions of less than 12 cm by less than 4 cm, such as less than 10 cm by less than 3 cm or from 12 to 4 cm by from 1 to 4 cm.

In some embodiments, the housing is made from a medically acceptable polymer, such as poly (lactic-co-glycolic acid), poly (ethylene vinyl acetate), polystyrene, polypropylene, poly vinyl chloride, polyethylene, polyurethane, polycarbonate, polyethylene terephthalate, polyetheretherketone or combinations thereof.

In preferred embodiments the housing is made from a flexible, medically acceptable polymer such as poly(ethylene vinyl acetate), polystyrene, polypropylene, poly vinyl chloride, polyethylene, polyurethane, polycarbonate, polyethylene terephthalate, polyetheretherketone or combinations thereof. In one embodiment the housing is made from poly (lactic-co-glycolic acid), poly (ethylene vinyl acetate) or polyurethane. In one embodiment the housing is made from poly(ethylene vinyl acetate).

In one embodiment, the housing is made from a medically acceptable, biodegradable polymer. Examples of a medical grade biodegradable polymer include poly (lactic-co-glycolic acid), polycapralactone, poly (lactic acid), starch, cellulose, poly (glycolic acid), poly(vinyl alcohol) or combinations thereof. Constructing the housing from a medically acceptable, biodegradable polymer allows the device to be implanted in locations where the permanent presence of a device is not desirable, but where further surgery to remove the device would be impractical or potentially harmful to the patient's health, e.g. the brain. The benefit of this embodiment is that over time the device will disintegrate and therefore once the drug composition has been transferred and the catheter removed, no further action is needed.

By medically acceptable polymer it is meant any polymer approved for surgical implants by a national regulatory body, such as the Food and Drug Administration, the Medicines and Healthcare Products Regulatory Agency, the European Medicines Agency, the Therapeutic Goods Administration, Pharmaceuticals and Medical Devices Agency.

Flexibility may be measured on the Shore Durometer Type A scale according to ASTM D2240. In some embodiments, the flexible polymer has a score of less than 60 on the Shore Durometer Type A scale.

In some preferred embodiments the housing has a rectangular cross section and is made from poly(ethylene vinyl acetate). This combination of shape and flexible, medically acceptable polymer improves the ease with which the implantable device can be implanted, because the device can adapt to the implant location, making it easier for a medical professional to attach the device to the body and reducing the risk that it will detach from the location following attachment.

In some embodiments, the housing is formed of two separate parts that can be joined together by a push fit or similar join to define the enclosed space and produce the complete implantable device of the invention. In one such embodiment, the first part of the housing is provided with the catheter port and the second part is provided with the permeable release zone and the attachment portion. Other such arrangements would be apparent to one skilled in the art.

Once transferred to the enclosed space in the form of a drug composition in fluid form, the drug passes through the permeable release zone to the treatment site. The permeable release zone is a zone in the device through which the drug is able to pass from the enclosed space into the patient's body. In some embodiments the drug passes through the permeable release zone together with one or more further components of the drug composition in fluid form. In some embodiments, both solid and fluid components of the drug composition can pass through the permeable zone. In other embodiments, only solid or only fluid components can pass through the permeable zone.

It will be appreciated that a concentration gradient may act to cause the drug to pass through the permeable release zone into the patient's body and to the treatment site.

The permeable release zone may itself be an opening and the drug may pass through this opening. The permeable release zone may be provided with perforations, and the drug may pass through these perforations in the permeable release zone. The permeable release zone may be a membrane extending across one or more openings in the housing, and the drug may be drawn into and diffuse through the membrane. It may be that the permeable release zone is a membrane and the membrane has perforations, and the drug may pass through the perforations in addition to or instead of the drug being drawn into and diffusing through the membrane.

The skilled person will therefore understand that the permeable release zone may be configured in a number of different ways, but all of these provide routes by which the drug can exit the enclosed space and reach the treatment site which is adjacent to the permeable release zone.

In some embodiments the permeable release zone is configured to release the drug in an immediate manner, meaning that the progress from the enclosed space to the body is not slowed by the permeable release zone.

In other embodiments the permeable release zone is configured to release the drug in a sustained manner, meaning that the permeable release zone slows the release of the drug from the enclosed space, such that release takes place over hours, days, weeks, months or years.

It will be appreciated that the skilled person can make choices as to the extent to which they want the permeable release zone to cause a delay in the drug reaching the treatment site. A delay due to the permeable release zone is optional. As discussed above, a benefit of the present invention is that there is control of the timing of the drug being released, because this cannot occur before the drug composition is transferred to the enclosed space. Optionally, in addition there may be further control built in due to the permeable release zone being configured to cause a delay in the release of the drug from the enclosed space into the patient's body.

In some embodiments the permeable release zone is configured to release the drug over the course of 24 hours or more, such as 48 hours or more, or 72 hours or more: e.g. 7 days or more, or 10 days or more, or 14 days or more. In some embodiments, the permeable release zone is configured to release the drug over a period of from 10 to 21 days, such as from 10 to 16 days.

In some embodiments, the permeable release zone is a section of the housing comprising one or more perforations, such as from 1 to 10 perforations, or 1 to 6 perforations, or 1 to 4 perforations. The perforations may be any shape, e.g. they may be holes with a circular cross section, or they could be elongate slits These perforations may each independently have a diameter or longest dimension of from 0.1 to 3 mm, such as from 0.5 to 2 mm or from 1 to 1.5 mm. The perforations may be pores.

When perforations, such as pores, are present in the membrane, the drug may pass through these perforations, e.g. pores, in addition to diffusing through the membrane or instead of diffusing through the membrane.

In some preferred embodiments, the permeable release zone is a membrane, such as a polymer membrane, in particular a membrane extending over a window in the housing. The drug can pass through the membrane. In some embodiments the membrane is made from the same material as the housing. In other embodiments the membrane is made from a different material to the housing. Where the housing is made from a medically acceptable, biodegradable polymer it is preferred that the membrane should also be made from a medically acceptable, biodegradable polymer (as defined above).

When the permeable release zone is a polymer membrane, in use the drug may be drawn into and diffuse through the polymer of the membrane to reach the treatment site which is adjacent to the permeable release zone.

In some embodiments the membrane is a polymer membrane made from poly (lactic-co-glycolic acid), poly (ethylene vinyl acetate), polystyrene, polypropylene, poly vinyl chloride, polyethylene, polyurethane, polycarbonate, polyethylene terephthalate, polyetheretherketone, polycapralactone, poly (lactic acid), starch, cellulose, poly (glycolic acid), poly(vinyl alcohol) or combinations thereof.

Permeable membranes are known in the art. The material for the membrane, the thickness of the membrane, the number of perforations (e.g. pores) in the membrane, and the size of perforations (e.g. pores) in the membrane can each independently be controlled. These parameters can be adjusted to ensure that the drug as provided in the enclosed space is released, e.g. over a sustained period, to the treatment site. The desired rate of release will depend on the drug to be delivered and the medical condition of the patient. The skilled person would be able to determine the appropriate membrane characteristics to achieve the release rate required. However, further details are provided below.

A membrane can usefully be manufactured using techniques including 3D printing, injection moulding, hot melt extrusion or solvent casting. The invention is not limited to the method of manufacture for the membrane.

The thickness of the membrane can impact the rate of release and the stability of the device. If the membrane is too thick, the release of the drug may be too slow for the desired therapeutic effect. If the membrane is too thin there is a risk that the membrane will burst, releasing the drug immediately. Where the permeable release zone is a membrane, the membrane thickness is usefully from 0.25 mm to 5 mm, preferably from 0.5 mm to 4 mm, such as from 0.75 mm to 3 mm, e.g. from 1 mm to 3 mm, or from 0.75 mm to 2 mm, e.g. from 0.75 mm to 1.5 mm or from 1 mm to 2 mm. In one preferred embodiment the membrane has a thickness of from 0.75 to 1.25 mm.

The number of perforations in the membrane can also impact the rate of sustained release. In one embodiment, the perforations are pores and the porosity of the membrane can also impact the rate of sustained release. The more perforations, e.g. pores, that are present in the membrane, the quicker the drug is released from the device. In some embodiments where the permeable release zone is a membrane, the membrane contains from 1 to 100 perforations, e.g. pores, such as from 1 to 10, from 2 to 8, or 4 to 6 perforations, e.g. pores.

The size of the perforations in the membrane can also impact the rate of sustained release. In some embodiments the perforations, e.g. pores, each have a diameter or largest dimension of 0.1 to 10 mm, such as from 2 mm to 5 mm, from 2.5 mm to 4.5 mm, or from 3 mm to 4 mm. For example, the membrane may have from 1 to 10, such as from 4 to 6 pores, wherein the pores each independently have a diameter of from 2 mm to 5 mm, such as from 3 mm to 4 mm.

The size of the permeable release zone also impacts the rate of release. The area of the permeable release zone, e.g. permeable membrane, will vary depending on the implantation site. In one embodiment the maximum dimension of the permeable release zone, e.g. permeable membrane, is in the range of from 2 mm to 30 mm, such as from 5 mm to 25 mm or from 5 mm to 20 mm. Suitable sizes include but are not limited to: 5 mm×5 mm, 5 mm×10 mm, 5 mm×10 mm, 10 mm×20 mm, 20 mm×20 mm, 10 mm×10 mm, 20 mm×20 mm.

In one embodiment the membrane is made from poly(ethylene vinyl acetate) and has a thickness of from 0.8 to 1.2 mm. In one such embodiment, the membrane does not include any perforations.

As the skilled person will appreciate, with 3D printing it is possible to set an infill value for the 3D printed product, reflecting the fact that gaps can be left in the printing pattern which are not filled with polymer. Thus, using 3D printing enables a choice of infill values for a polymer membrane. This contrasts with traditional techniques, such as injection moulding, hot melt or solvent casting, where it is not possible to leave such gaps and therefore the resulting membrane would always have 100% infill.

In embodiments where the membrane is manufactured using 3D printing, the skilled person will appreciate that they should select a suitable infill value. The infill value should not be too low, because this impacts structural integrity (there is too much of the membrane that comprises gaps, rather than being formed from polymer) and there can be a risk that the membrane will burst: in this regard it is preferred that the infill of the membrane is at least 50%. However, there is the opportunity, should they wish, to further control the rate of drug release by controlling the infill value selected. In some embodiments the skilled person may choose to have less than 100% infill, because this may assist with the release of the drug being quicker. Clearly whether this is desirable will depend on the drug and the desired therapeutic effect. In one embodiment where the permeable release zone is a membrane, the infill of the membrane is usefully from 50% to 100%, such as from 60 to 100% or from 70 to 100%. In some embodiments the membrane has an infill of 100%. In some embodiments the infill of the membrane is from 70% to 95%, e.g. from 75% to 90%.

In some embodiments, the membrane is permanently fixed to, or forms part of, the housing of the implantable device. In an alternative embodiment, the membrane is releasably attachable to the implantable device. The alternative embodiment allows the membrane to be changed. This allows the implantable device to be adjusted to achieve a desired release rate or to be suitable for a specific drug composition.

In some embodiments, the implantable device comprises a sponge contained within the enclosed space. In some embodiments the sponge is the same size as the enclosed space, thereby filling the entirety of the enclosed space, whilst in other embodiments the sponge is smaller than the enclosed space, thereby filling only part of the enclosed space.

In some embodiments, the drug composition is in liquid form and the implantable device comprises a sponge contained within the enclosed space. When the drug composition in liquid form is transferred to the enclosed space, the sponge absorbs the drug composition in liquid form and holds it within the enclosed space. Both the drug and other components of the drug composition such as a liquid carrier or diluent will be absorbed by the sponge in the enclosed space. Effectively, the sponge containing the drug composition can be considered as a solid content for the device. This arrangement is therefore also less likely to lead to pressure on external walls of the device, and therefore this option also reduces the risk of bursting.

Where present, the sponge should suitably be biocompatible and medically acceptable for implantation into a patient's body. In some embodiments, the formulation of the sponge comprises gelatin. In some embodiments the sponge is a gelfoam sponge comprising or consisting of gelatin.

In some embodiments the sponge has a pore size of 50 to 100 micrometres, such as from 60 to 90 micrometres, from 65 to 85 micrometres or from 70 to 80 micrometres. In some embodiments the sponge has a porosity of from 100 to 150%, such as from 110 to 140% or from 120 to 130% or from 130 to 140%. In some embodiments the sponge has a hardness of from 2000 to 3500 g when dry and of from 500 to 1500 g when wet. In some embodiments the sponge has a swelling ratio of from 10:1 to 15:1, such as from 11:1 to 14:1 or from 12:1 to 13:1. In some embodiments the sponge has an elasticity of from 55 to 90% when dry, such as 65 to 85% or 75 to 80%, and an elasticity of from 3 to 18% when wet, such as from 7 to 14% or from 10 to 13%. Methods of determining the properties of sponges are provided in the examples section.

The housing of the implantable device is provided with an attachment portion. The attachment portion allows the medical professional to fix the implantable device within the body of the patient, such that it does not travel within the body once implanted. By fixing the implantable device within the body using the attachment portion the medical professional can ensure that the permeable release zone is located adjacent to the treatment site, e.g. in physical contact with the treatment site.

In some embodiments the attachment portion is a skirt extending from the housing, optionally wherein the skirt is formed from a polymer membrane. This skirt may form a complete ring around the perimeter of the housing, or may include one or more spaces so as to be an incomplete ring, e.g. the skirt may comprise a series of tabs arranged in a ring around the housing. In some embodiments the attachment portion has a thickness of from 0.5 mm to 4 mm, such as from 1 mm to 3 mm, from 1 mm to 2 mm or from 0.75 to 1.25 mm.

In some embodiments the attachment portion is formed from the same material as the membrane that forms the permeable release zone. In some embodiments the attachment portion is part of the membrane that forms the permeable release zone.

The attachment portion, e.g. skirt, may be fixed at the desired location within the body using sutures. Alternatively, the attachment portion, e.g. skirt, may be glued at the desired location within the body.

Other means of attaching the attachment portion, e.g. skirt, at the desired location within the body are known and would be apparent to those skilled in the art.

In other embodiments the attachment portion comprises one or more ties that are releasably attached to, or form part of, the housing of the device. The ties can be fastened around a suitable component of the body of the patient, such as a bone or part of an organ, to hold the implantable device in position at the desired location within the body.

The implantable device comprises a catheter port. Catheter ports are well known in the field of the invention. Catheter ports provide a port into which a catheter can be inserted to provide a fluid tight seal, allowing fluid to pass from the catheter and through the catheter port. In the present invention, the catheter port provides an access point, through which a drug composition can be transferred to the enclosed space via a catheter.

Catheters are well known in the field of the invention, and the skilled person would be able to identify a suitable catheter for use in the invention.

The implantable device may be provided in the form of a kit, together with a catheter.

In embodiments where the permeable release zone is a membrane that is releasably attachable to the housing, the kit may be further provided with one or more membranes, such as two or more, or four or more, or 10 or more membranes. Where the kit comprises two or more membranes, these membranes suitably have different characteristics, such that they are suitable for the delivery of different drugs and/or over different time scales. Thus, the medical professional can select a suitable membrane for use.

The invention also provides a method of facilitating drug administration to a patient. This can in particular be used following surgery, such as surgery to remove one or more tumours. The method comprises the steps of: a) providing an implantable device for drug delivery according to the invention: b) placing the device within the body of the patient; and c) attaching the device, using the attachment portion, at a location such that the release zone is adjacent to a treatment site. In one embodiment, the treatment site is a resection site, e.g. a surgical margin.

The method may further comprise: i) providing a catheter; and ii) attaching the catheter to the catheter port: wherein steps i) and ii) can be carried out before or after any of steps a) to c). In one embodiment, step i) is carried out before step b) and step ii) is carried out before or after step b).

The method may further comprise:
d) providing a drug composition in fluid form to the enclosed space, via the catheter and the catheter port; and
e) allowing the drug to be released to the treatment site via the permeable release zone.

In one embodiment there is a controlled delay period between step c) and step d). For example, the delay period may be a day or more, or a week or more, or two weeks or more, or three weeks or more, or four weeks or more.

Following the method of the present invention, the surgical opening may be closed using standard surgical techniques known in the art.

The time between attaching the device and providing the drug composition in the enclosed space is determined by the medical profession based on the specific circumstances of the patient. The medical professional can base their decision on when to transfer the drug composition into the enclosed space based on various factors such as whether the surgical wound has sufficiently healed, whether the patient is suffering from post-surgery complications, whether the patient needs to undergo further medical procedures, whether sufficient resources are available to support the patient once the drugs have been transferred or whether the patient desires a period from treatment for personal reasons. By controlling the time between attaching the device and providing the drug composition in the enclosed space, the medical professional is better able to achieve the best possible outcome for the patient.

In some embodiments, the drug composition is transferred immediately following closure of the surgical opening. This timing might be selected if the medical professional determines during surgery that immediate administration of the drug composition is essential to the patient's recovery, such that the risks associated with such a timetable are appropriate.

However, in preferred embodiments, the drug composition is transferred to the enclosed space at least 1 day after attaching the device, such as at least 3 days, at least 5 days, at least 7 days, or at least 14 days after. In some embodiments the drug composition is not transferred to the enclosed space for at least 21 days following the attachment of the device.

In some embodiments, the device may be re-usable. For example, after a first drug composition has been released to the treatment site via the permeable release zone, the device can be re-used to administer a second drug composition to the patient. This second drug composition in fluid form can be provided according to step d) as described above and release to the treatment site according to step e) above. The device can be cleaned before re-use, e.g., using a cleaning step whereby the enclosed space is washed out using a cleaning solution, such as water or saline, that can be provided via the catheter. In some embodiments the cleaning solution may be allowed to be released into the body via the permeable release zone. In other embodiments the cleaning solution may be removed, using standard drainage procedures, via the catheter.

The device could be re-used to administer two or more drug compositions, such as three or more, or four or more drug compositions. The drug compositions may be the same or different.

Although the drug composition that can be administered by using the implantable device of the invention does not form part of the invention, the device, kit and method are envisioned for the delivery of various types of drug composition and in particular chemotherapy drug compositions. Amongst these are the FOLFIRINOX regime comprising leucovorin, fluorouracil, ironitecan and oxaliplatin.

The drug composition for use in the device of the invention is in fluid form, such that it may flow through a catheter into the device. In particular, it is foreseen that the drug composition may be a solution of one or more drugs in a pharmaceutically acceptable carrier, or a suspension of one or more drugs in a pharmaceutically acceptable carrier. The use of an emulsion, e.g. water-in-oil or oil-in-water, can also be contemplated. However, the drug composition comprising one or more drugs may be in any other fluid form, e.g. it may be a free-flowing powder or it may be a semi-solid form such as a gel, cream or paste.

In general, the drug composition may comprise one or more drugs in combination with a pharmaceutically acceptable carrier or diluent.

The drug composition may of course optionally include further pharmaceutically acceptable components, as well known in the pharmaceutical field: e.g. pharmaceutically acceptable adjuvants, fillers, buffers, preservatives, anti-oxidants, lubricants, stabilisers, solubilisers, and/or surfactants (e.g., wetting agents).

Suitable carriers, diluents, excipients, etc. can be found in standard pharmaceutical texts, for example, Remington's Pharmaceutical Sciences, 18th edition, Mack Publishing Company, Easton, Pa., 1990; and Handbook of Pharmaceutical Excipients. 5th edition, 2005.

### DETAILED DESCRIPTION OF THE DRAWINGS

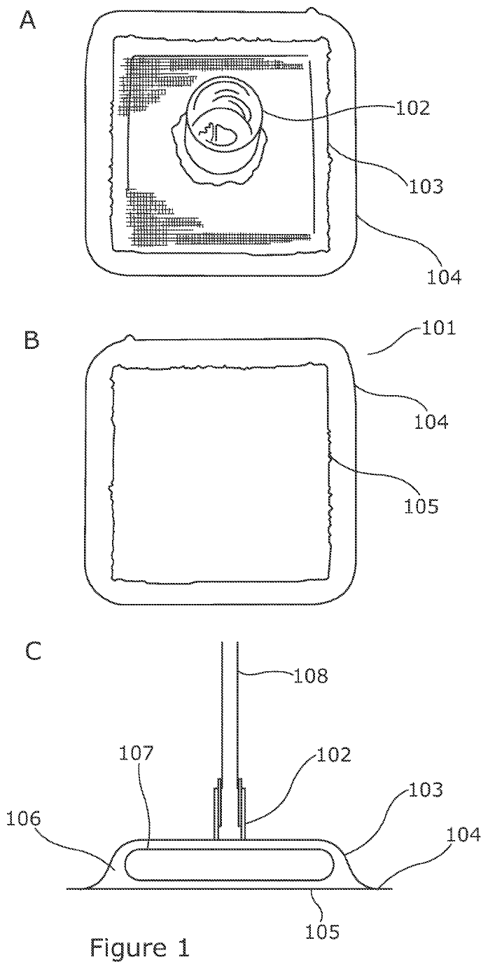

FIG. 1 shows an implantable device according to the invention. FIG. 1A is a view from a side of the device bearing the catheter port, FIG. 1B is a view from a side of the device comprising the permeable release zone and FIG. 1C is a cross section of the device along the passage defined by the catheter port.

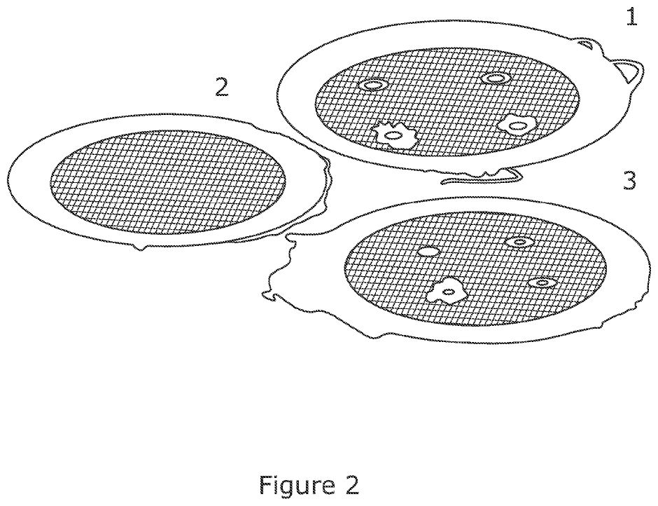

FIG. 2 shows three examples (1, 2, 3) of the membrane that may form the permeable release zone used in the device of the invention.

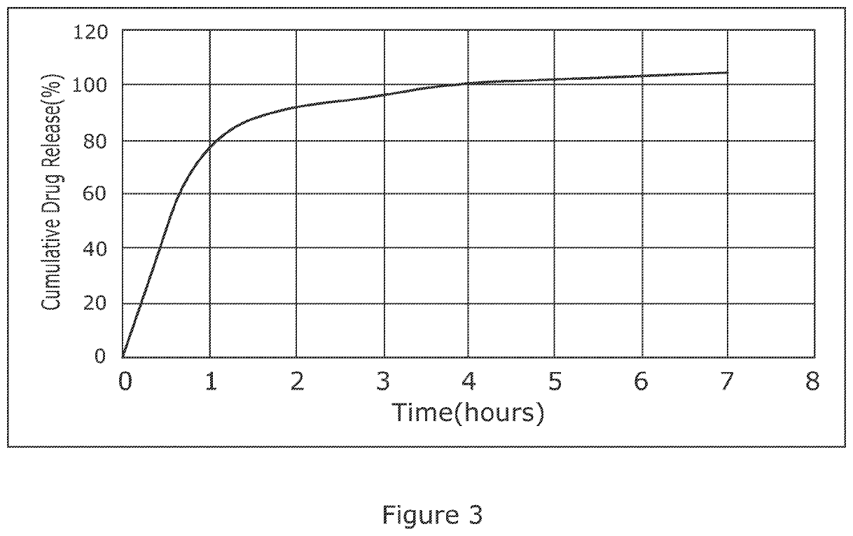

FIG. 3 shows the release profile of oxaliplatin from an exposed sponge (comparative).

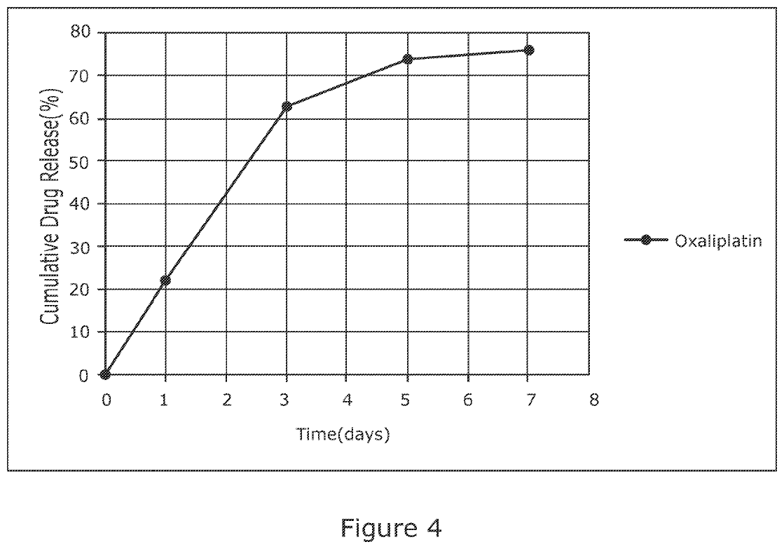

FIG. 4 shows the release profile of oxaliplatin from an implantable device according to the invention.

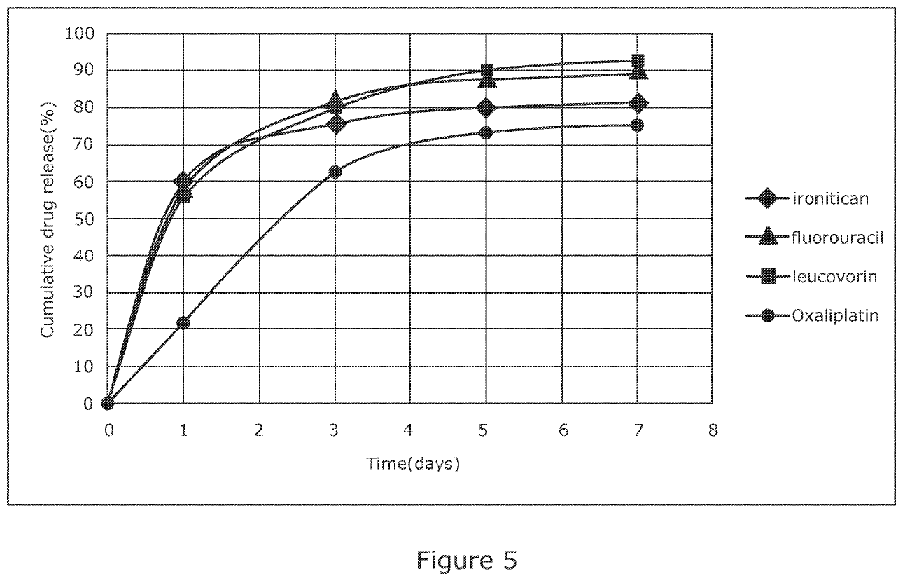

FIG. 5 shows the release profile of FOLFIRINOX from an implantable device according to the invention.

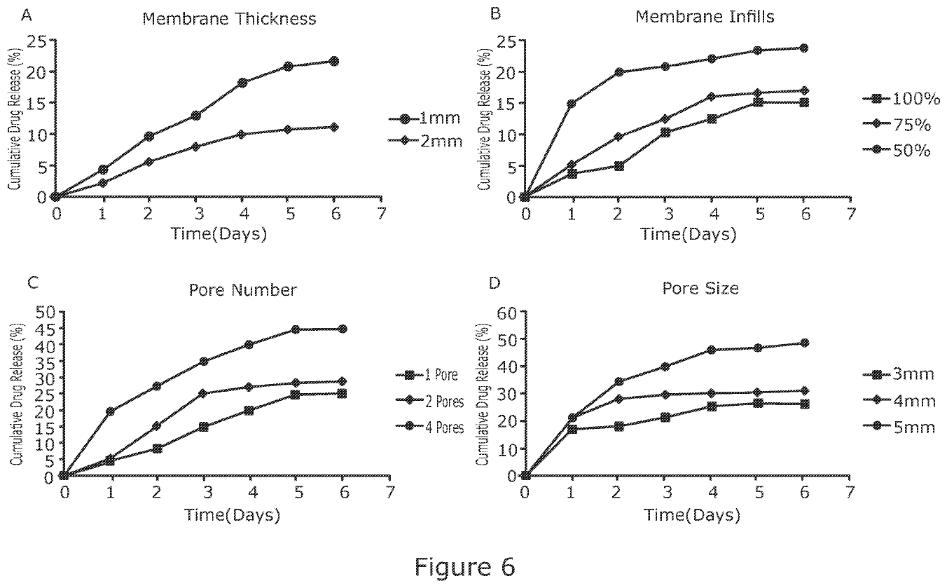

FIG. 6 shows the release profiles of irinotecan from an implantable device according to the invention when provided with different membranes as the permeable release zone.

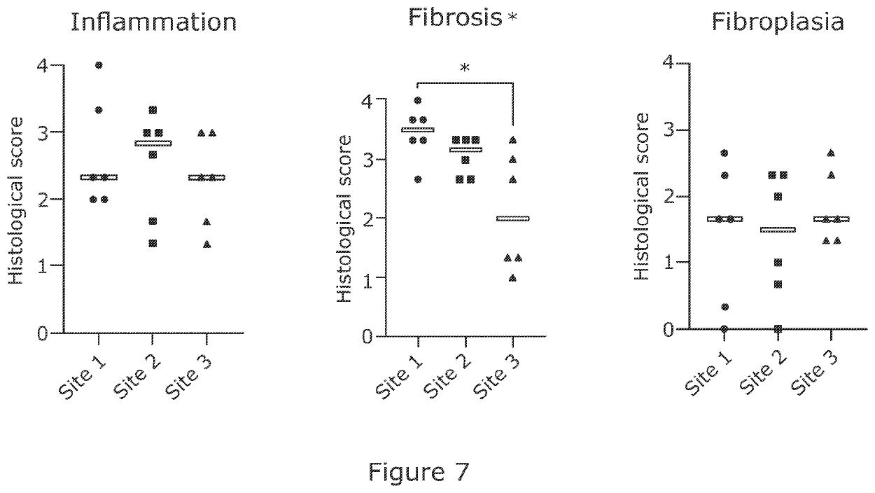

FIG. 7 shows dot plots illustrating an overall summary of the histological (Inflammation, Fibrosis and Fibroplasia) findings for implantation of a device according to the invention in pigs.

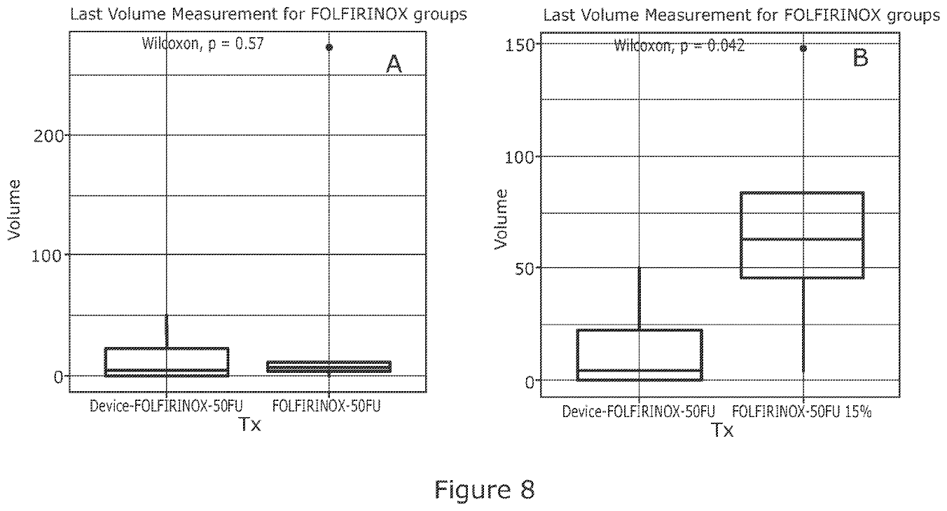

FIG. 8 shows a statistical comparison of 28-day tumour volume between administration via a device according to the invention with 15% FOLFIRINOX and the systemic administration of the 100% (A) and 15% (B) FOLFIRINOX dose.

Referring firstly to FIGS. 1A-C of the accompanying drawings, these show an implantable device 101 according to the present invention, comprising a housing 103 defining an enclosed space 106. Within the enclosed space is optionally housed a sponge 107 that, when present, partially fills the enclosed space. The housing 101 is provided with a catheter port 102 capable of providing fluid access to the enclosed space; and an attachment portion 104 for attaching the housing at a location within the body of the patient. The attachment portion 104 is a fringe of polymer membrane suitable for sewing or gluing to the body of the patient. The device 101 is further provided with a permeable release zone 105. The permeable release zone 105 is positioned on the face of the housing opposed to the face bearing the catheter port 102. The permeable release zone is a membrane, usefully a polymer membrane. In one aspect the invention is a kit comprising the implantable device 101 and a catheter 108.

Referring to FIG. 2, three examples of membranes for use as permeable release zones are shown. Membrane (1) is provided with four pores each with a diameter of 5 mm. Membrane (2) has no pores. Membrane (3) has four pores each with a diameter of 3 mm.

### EXAMPLES

### Texture Analysis

The hardness and elasticity of sponges were assessed using TA-XT2 Texture Analyzer (Stable Micro Systems, Haslemere, UK). The test that was chosen is “make up sponges” for firmness and springiness determination. The mode was “Measure Force in Compression”, pre-test speed was 1.0 mm/s, test-speed 1.0 mm/s, post-test speed was 10.0 mm/s, target mode was Distance, hold time was 30 s, strain was 50%. A plot can be obtained which depicts a Force-Time curve displaying the characteristics of a sponge hardness and elasticity test.

The probe compresses the sample until it reaches 50% of the product height. It remains at this distance for 30 seconds before withdrawing from the sample and returning to its original position.

Hardness is defined as the force (in grams) required to compress a product by a predetermined distance, such as 50%. To calculate the elasticity property, take the force after 25 seconds, divide it by the maximum force, and multiply by 100 percent. F25 multiplied by 100 equals Fmax. The product is more like a spring the closer the resulting value is to 100%.

### Porosity

Sponge porosity was assessed by the liquid displacement method. In this experiment, absolute ethanol was used because it permeates through the sponge but doesn't cause any swelling or shrinkage. The dry sponge was weighed first, then immersed in 10 ml ethanol (V1) and degassed for 5 min with a vacuum pump. The total volume of ethanol and sponge was recorded as V2. The soaked sponge was removed, and the remaining volume of ethanol was recorded as V3. The porosity was calculated using the equation:

$$ε 1 ( \% ) = ( V 1 - V 3 / ( V 2 - V 3 ) \times 100$$

### Swelling Ratio

The swelling ability of a sponge was measured by immersing the pre-weighed sponge (W0) into deionized water for 1 hour at room temperature. The soaked sponge was removed and re weighed (W1) after gently removing the excess water by filter paper. The swelling ratio was calculated using the equation:

$$ε 2 ( \% ) = ( W 1 - W 0 ) / W 0 \times 100$$

### Pore Size

Pore size can be determined using Scanning Electron Microscopy (SEM) analysis.

The morphology of the sponges was examined using SEM (Jeol 6060, Oxford Inca EDS). Before the examination, the samples were sputter coated with gold and placed in a SEM vacuum chamber.

### Drug Release Rate

Drug release rate was measured using 50 mL of release media consisting of 50:50 DMSO:water to ensure that all drugs in the composition remained under sink conditions. Each device was suspended above the release media in a 50 mL sealed Duran bottle with only the permeable release zone in contact with the release media. The Duran bottle was subsequently placed in a shaking incubator at 37° C. and 60 RPM. 5 mL samples of release media were removed and replaced with 5 mL of fresh media either every day for 6 days or at day 1, 3, 5 and 7. The samples were analysed for drug concentration using HPLC.

### Example 1

Release of the drug oxaliplatin by (i) an exposed sponge and (ii) a device according to the present invention was compared. The sponge was a gelatin sponge exposed to 50 mL of release media consisting of 50:50 DMSO. The sponge had the proportions 5 mm×10 mm×10 mm. The implantable device according to the invention had a membrane as the permeable release zone, the membrane was a 3D printed poly (ethylene vinyl acetate) membrane being 1 mm thick, with no pores and a 75% infill. The permeable release zone had a surface area of 100 mm². The implantable device according to the invention contained a sponge within the enclosed space. Drug release was determined according to the methodology outlined above.

FIG. 3 shows the release rate of the drug oxaliplatin from the exposed sponge. Release was complete within 7 hours.

FIG. 4 shows the release rate of the drug oxaliplatin from the implantable device according to the invention. Release was not complete at 7 days (168 hours). Release plateaued at 75% after 5 days.

The implantable device according to the invention therefore provides better sustained release of the drug oxaliplatin compared to an exposed sponge.

### Example 2

Release of the drug components of the FOLFIRINOX regimen from a device according to the invention was investigated. The implantable device according to the invention had a membrane as the permeable release zone, the membrane was a 3D printed poly (ethylene vinyl acetate) membrane being 1 mm thick, with no pores and a 75% infill. The permeable release zone had a surface area of 100 mm². The implantable device according to the invention contained a sponge within the enclosed space. Drug release was determined according to the methodology outlined above.

FIG. 5 shows the release of the drugs ironitecan, fluorouracil, leucovorin, and oxaliplatin was measured over 7 days. The drugs were released but the release of the drugs began to plateau after three days and was still not complete after 7 days. The implantable device therefore provides for sustained release of multiple drugs.

### Example 3

The release of the drug irinotecan over 6 days through various membranes for use as the permeable release zone was investigated, to show how the device of the invention provides the skilled person with control over the release profile for the drug, by varying properties of the membrane.

Membranes with a thickness of 1 mm or 2 mm were investigated. The results are shown in FIG. 6A. It was found that the drug was released faster through the membrane with a thickness of 1 mm than through the membrane with a thickness of 2 mm. Therefore, the skilled person can vary the thickness of the membrane to achieve faster or slower drug release, as desired.

3D printed membranes with an infill of 50%, 75% and 100% were investigated. The results are shown in FIG. 6B. It was found that as the percentage infill increased, the release rate slowed. Therefore, the skilled person can vary the infill of a 3D printed membrane to achieve faster or slower drug release, as desired.

Membranes with one, two or four pores were investigated. The results are shown in FIG. 6C. The pores each had a diameter of 3 mm. It was found that as the number of pores increased the rate of release also increased. Therefore, the skilled person can vary the number of pores in a membrane to achieve faster or slower drug release, as desired.

Membranes with pore sizes of 3 mm, 4 mm and 5 mm were investigated. The results are shown in FIG. 6D. The membranes each had 4 pores. It was found that as pore size increased the rate of release also increased. Therefore, the skilled person can vary the size of the pores in a membrane to achieve faster or slower drug release, as desired.

Despite the variation between each of the membranes, it was found that each membrane investigated provided a useful sustained release of the drug irinotecan from the enclosed space. Therefore, all of the membranes tested can be used in the present invention.

### Example 4

A device according to the invention was evaluated for safety using pig models.

Histology shown in FIG. 7 of the anastomosis indicated that the implant with no added chemotherapy (Site 2) was associated with less fibrosis than the no implant control (Site 1) with the lowest fibrosis after administration of FOLFIRINOX through the device (Site 3). There was no evidence of anastomotic failure.

Device implantation and the local delivery of low dose (15%) FOLFIRINOX does not cause toxicity or anastomotic leaks in pigs.

### Example 5

A device according to the invention was evaluated for efficacy using mouse models.

Devices according to the invention were implanted onto the resection margin of mice with a Patient Derived Xenograft (PDX) pancreatic tumour and compared to 100% and 15% systemic doses.

The 100% systemic treatment group had a statistically similar (P=0.57) day 28 tumour volume compared to the 15% local treatment (device) group as shown in FIG. 8A. However, out of the six mice in the device group only two (33.3%) had tumour tissue present after 28 days, while in the 100% systemic treatment group all six mice (100%) had tumour tissue present.

Furthermore, one mouse died in the 100% systemic treatment group as a result of treatment toxicity. The 15% device group had a statistically significant (P=0.042) reduction on the day 28 tumour volume when compared to the 15% systemic treatment group as shown in FIG. 8B, while all six (100%) mice in the systemic treatment group had tumour tissue present after 28 days.

Histological analysis of the tumour tissue from each treatment group demonstrated 20 to 30% stroma cells and approximately 40% fibrosis in the 15% device group, while the 100% systemic treatment group showed between 40 and 80% stroma cells, 20 to 30% fibrosis, with up to 90% necrosis. The histology of the 15% systemic treatment group demonstrated a large amount of infiltrating stroma tissue, with approximately 10 to 20% fibrosis. Pancreatic cancer is characterised by extremely dense stroma tissue, while the fibrosis is typically induced by chemotherapy.

The reduced stroma tissue and increased fibrosis in the 15% device group compared to the 15% and 100% systemic treatment groups demonstrated the improved efficacy of the localised (device) delivery of low dose FOLFIRINOX, with more of the dose reaching the tumour tissue compared to systemic FOLFIRINOX.

### CONCLUSIONS

The device according to the invention provides allows the medical professional to accurately control where a drug composition will be administered within the body. A specific location can be selected and in this regard the device is placed near that location, with the permeable release zone adjacent to the selected treatment site, e.g., resection site.

The device also allows the medical professional to control when the drug composition will be administered to the specific location within the patient. The medical professional can take into account factors that cannot be determined before the surgery is complete and decide when the patient is ready to receive the drug. The patient will not receive the drug until after the enclosed space has been provided with the drug composition, and therefore the medical professional can build in a controlled delay to take account factors such as how quickly the patient is recovering from the surgery.

The medical professional also has the ability to further optimise the release profile for the drug to the patient, by choosing and varying characteristics of the permeable release zone. The medical professional can take into account factors such as the type of drug, the patient's characteristics and the disease characteristics and assess whether the drug should be provided in immediate release or sustained release form, and if so how quickly or slowly the drug should be released. The medical professional can then select and control properties of the permeable release zone, such whether a membrane is used: the material used: the thickness used: the number of perforations, e.g., pores, and the size of perforations, e.g. pores.

1. An implantable device for drug delivery to a patient, the device comprising:
a housing defining an enclosed space;
a catheter port that provides fluid access to the enclosed space;
a permeable release zone that permits drug release from the enclosed space; and
an attachment portion for attaching the housing at a location;
such that, in use, the implantable device can be placed within the body of the patient and attached, using the attachment portion, at a location such that the release zone is adjacent to a treatment site, and whereby a drug composition in fluid form can be provided to the enclosed space via a catheter and the catheter port, and the drug can be released to the treatment site via the permeable release zone.

2. The implantable device of claim 1, wherein the housing is constructed from a flexible, biocompatible polymer selected from the group consisting of: poly (lactic-co-glycolic acid), poly (ethylene vinyl acetate), polystyrene, polypropylene, poly vinyl chloride, polyethylene, polyurethane, polycarbonate, polyethylene terephthalate, polyetheretherketone, polycapralactone, poly (lactic acid), starch, cellulose, poly (glycolic acid), poly(vinyl alcohol) and combinations thereof.

3. The implantable device of claim 1 or claim 2, wherein the attachment portion comprises a skirt extending from the housing, optionally wherein the skirt is formed from a polymer membrane.

4. The implantable device of claim 3, wherein the skirt is formed from the same material as the permeable release zone.

5. The implantable device of any one of claims 1 to 4, wherein the permeable release zone is formed from a polymer membrane, optionally wherein the membrane has a thickness of from 0.1 mm to 3 mm.

6. The implantable device of claim 5, wherein the membrane comprises one or more perforations, optionally from 1 to 10 perforations.

7. The implantable device of claim 6, wherein the perforations each independently have a diameter or largest dimension of from 0.1 to 5 mm.

8. The implantable device of any one of claims 1 to 7, wherein the device further comprises a sponge contained within the enclosed space.

9. The implantable device of claim 8, wherein the sponge is a medical grade sponge, optionally a gelatin sponge.

10. The implantable device of any one of claims 1 to 9, wherein the housing has a first face and second face, wherein the two faces are opposed to one another and spaced apart by at least one wall, the first face being provided with the release zone and the second face being provided with the catheter port.

11. A kit comprising (i) the implantable device as defined in any one of claims 1 to 10 and (ii) a catheter.

12. A method of facilitating drug administration to a patient, the method comprising the steps of:
a) providing an implantable device for drug delivery as defined in any one of claims 1 to 10;
b) placing the device within the body of the patient; and
c) attaching the device, using the attachment portion, at a location such that the release zone is adjacent to a treatment site.

13. The method of claim 12, wherein steps a) to c) are carried out during or after surgery on the patient, optionally wherein the surgery is a surgery to remove one or more tumours.

14. The method of claim 12 or claim 13, wherein the treatment site is a resection site.

15. The method of any one of claims 12 to 14, further comprising:
i) providing a catheter; and
ii) attaching the catheter to the catheter port;
wherein steps i) and ii) can be carried out before or after any of steps a) to c).

16. The method of claim 15, further comprising the steps of:
d) providing a drug composition in fluid form to the enclosed space of the housing, via the catheter and the catheter port; and
e) allowing the drug to be released to the treatment site via the permeable release zone.

17. The method of claim 16 wherein there is a delay period between step c) and step d), optionally wherein the delay period is a day or more, or a week or more, or two weeks or more, or three weeks or more, or four weeks or more, or six weeks or more.
